(19) United States
(12) Plant Patent Application Publication
(10) Pub. No.: US 2016/0381845 P1
(43) Pub. Date: Dec. 29, 2016

(54) BUXUS 'Clair Curtis'

(50) ;

(71) Applicant: Hugh Major McKittrick, Colts Neck, NJ (US)

(72) Inventor: Hugh Major McKittrick, Colts Neck, NJ (US)

(21) Appl. No.: 14/751,797

(22) Filed: Jun. 26, 2015

(51) Int. Cl. A01H 5/04

(52) U.S. Cl. CPC A01H 5/04
USPC 800/298

(57) ABSTRACT

A New and distinct BUXUS cultivar. The new cultivar is of benefit to the Ornamental plant industry. This cultivar possessing meritorious characteristics defining its plurality in appearance. This Boxwood shall here forth be known as Clair Curtis. The plant has a definite upright pyramidal form with 1 (one) central leader, with a radiating branch structure. The branches consisting of widely spaced internodes in an alternating pattern. The leafs are noticeably larger, than BUXUS Sempervirens Varder Valley or Newport Blue. The coloration on large, newly emerged leafs possess a blue opalesense similar to BUXUS “Varder Valley”. The adult foliage maintaining size and possessing a dark green color. Fragrance is not present in this cultivar. These traits have been observed over the past several winters to have survived temperatures of −2 (minus Two) degrees Fahrenheit in an outdoor, exposed siting. This BUXUS after enduring these various winters was able to maintain a high rate of asexual propagation without distortion.

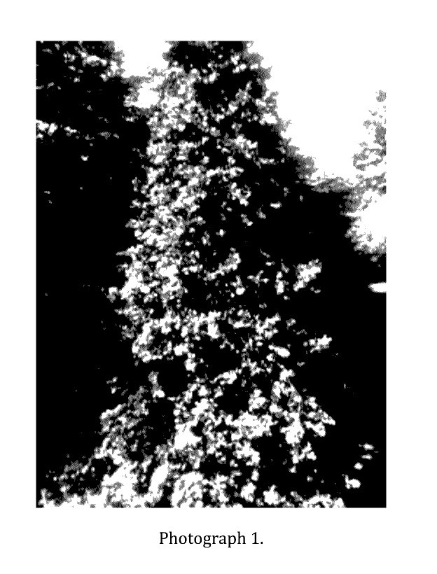

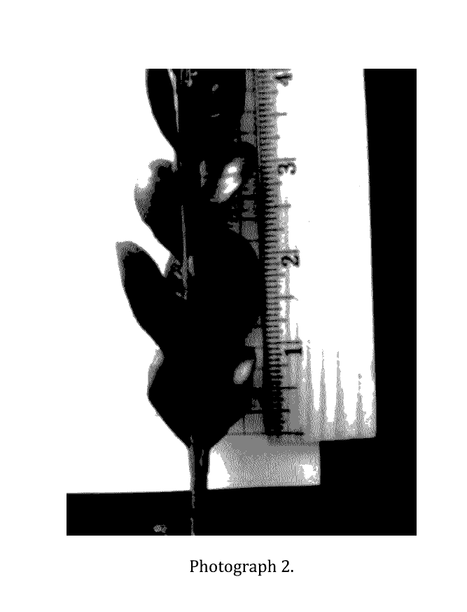

The continued preservation and subsequent use of this plant will benefit the the ornamental plant industry.

This new cultivar possesses naturally occurring qualities that distinguish it from related cultivars. The plant is pyramidal in form, with a central leader, with horizontal branching. This feature has naturally survived and retained form in all observed snow loads. The adult foliage color reflects parentage of BUXUS Sempervirens, with juvenile foliage having a noticeable blue matte opalescent on the top surface of the leaf. Comparision of adult and juvenile Leaf color and size are noticeably and measurably larger than the non-patented Buxus Sempervirens” Newport Blue”. The grow habit is broader at the base than BUXUS Dee Runk or Buxus pyramidalis (both plants non-patented) and is distinctly different in most other characteristics. These aforementioned characteristics are distinguishable from the patented variety BUXUS Katerberg. Foliage coloration and quality has been retained, while withstanding −2 (minus two degrees) Fahrenheit. Since the 1994 founding of this plant it has exhibited a natural resistance to pests commonly associated with the Species. Growth habits have remained constant. Rate of growth has been reliably 6-8 (six to eight) inches annually.

The combination of unique and meritorious features of this plant indicates it to be used as a specimen or hedge planting. The ornamental qualities of this newly discovered plant provide visual interest. This plant has expressed attractive traits exhibited as a course and rugged textural quality. Juvenile leaf growth possessing a blue opalesents persisting into the hardening off stage of development. The large evergreen leafs, in opposing sets, on widely spaced internodes arranged in a mildly alternating pattern of approximately 20 degrees from adjacent internodes. The strong annual growth rate allows for a multitude of applications. Superior performance in snow load conditions has been annually recognized. The plant has not been observed to poses any distinguishable fragrance. This new cultivar will be of beneficial use and possess traits with widespread benefits to the ornamental plant industry.

### BRIEF DESCRIPTION OF PHOTOGRAPHS

The accompanying color photographs show this new plant for the purpose of general and specific observations. Colors in the photographs may be different than the written description.

Photograph 1: Adult shown in spring. Photograph taken in observation citing in Colts Neck, N.J.

Photograph 2: The cut portion of annual growth shown on graph paper with ruler on edge of paper. Graph paper of ¼ inch scale. Ruler is of U.S. inch standards.

### DETAILED BOTANICAL DESCRIPTION

The subsequent description of the new plant is of a 1994 planting in Colts Neck, N.J. The details contained within have been repeatedly observed to have expressed these identifiable details. The colors referenced are standards set by The Royal Horticultural Society (R.H.S.) colour chart, unless otherwise noted.
Type: Broad leafed, pyramidal shaped evergreen shrub for ornamental landscape use.
Botanical classification: BUXUS hybrid of unknown parentage. To be known as ‘Clair Curtis’
Growth habit: Medium growing, full bodied, course and rugged foliage and naturally forming pyramidal profile.

It is claimed:

1. A new distinct Buxus hybrid plant expressing the following features:
Naturally manifests as an upright pyramidal form
Consistent and habitual uniform color retention in winter
Spring growth possessing opalescent blue qualities
Void of fragrance
Reproduces true and readily
Substantially as illustrated and described herein.
